(19) United States
(12) Patent Application Publication
(10) Pub. No.: US 2025/0082841 A1
(43) Pub. Date: Mar. 13, 2025

(54) WEARABLE MEDICAL DEVICE FOR ADJUSTING MEDICATION DELIVERY

(71) Applicant: INSULET CORPORATION, Acton, MA (US)

(72) Inventors: Rangarajan NARAYANASWAMI, Weston, MA (US); Jakob MORROW, Lawrence, MA (US); Jack THOMAS, Arlington, MA (US)

(21) Appl. No.: 18/828,725

(22) Filed: Sep. 9, 2024

Related U.S. Application Data
(60) Provisional application No. 63/582,041, filed on Sep. 12, 2023.

(51) Int. Cl. A61M 5/142; G16H 20/17

(52) U.S. Cl. CPC A61M 5/14244; G16H 20/17; A61M 2202/0007; A61M 2202/04; A61M 2205/3303; A61M 2205/3327; A61M 2205/3372; A61M 2205/50; A61M 2230/005; A61M 2230/201

(57) ABSTRACT

In an aspect, a wearable medical device for adjusting medication delivery is presented. The wearable medical device includes a drug delivery device. The wearable medical device includes at least a sensor configured to generate sensor data. The wearable medical device includes a processor in communication with the at least a sensor. The wearable medical device includes a memory communicatively connected to the processor. The memory contains instructions configured the processor to determine a submersion status based on the sensor data. The processor is configured to receive ambient temperature data from the sensor data. The processor is configured to calculate, based on the submersion status and the ambient temperature data, an adjusted delivery aspect of a medication for the user. The processor is configured to deliver the adjusted delivery aspect of a medication for the user.

### CROSS-REFERENCE TO RELATED APPLICATIONS

This application claims priority to and the benefit of U.S. Provisional Application No. 63/582,041, filed Sep. 12, 2023, the entirety of which is incorporated herein by reference.

### FIELD

The present subject matter generally relates to adjusting medication delivery. In particular, the present subject matter relates to a wearable medical device for modulating insulin delivery following showers and hot tub soaks.

### BACKGROUND

Patients with diabetes on insulin pumps may face glycemic fluctuations after hot showers/soaks in hot tubs/sauna baths. The higher temperature of the water may increase the rate of insulin absorption thus posing a hypoglycemic risk. The body strives to maintain an internal equilibrium in which the production of heat energy in the body is equal to the amount of heat loss to the external environment. This equilibrium is the easiest to maintain when the ambient temperature of the body falls into a zone called the Thermal Neutral Zone (TNZ). Within the TNZ, the body requires the least amount of energy to be pulled out of the bloodstream, i.e., glucose, to regulate and perform its necessary functions. However, as the ambient temperature of the body begins to fall out of the thermal neutral zone the expenditure of energy to heat the body, in the event of the ambient environment being cold, increases linearly as the temperature decreases. Likewise, when the body is exposed to an ambient environment which is hot, the expenditure of energy to cool the body down also increases linearly. Subsequently, when patients are exposed to ambient temperatures that are either in the extremes of being too hot or too cold, patients are prone to rapid and sudden decreases in blood glucose concentrations that if unexpected and unprepared for can lead to potentially life-threatening hypoglycemia. In the event of a patient swimming in a body of water that falls below the lower bound of the thermal neutral zone, the patient is going to experience an increased metabolic rate that will subsequently cause their blood glucose values to go down as the body attempts to heat itself. The reciprocal of this situation for a hot body of water is also true, as the patient's metabolic rate will also increase as the body attempts to cool itself down.

### SUMMARY OF THE DISCLOSURE

In an aspect, a wearable medical device for adjusting medication delivery is presented. The wearable medical device includes a drug delivery device. The wearable medical device includes at least a sensor configured to generate sensor data. The wearable medical device includes a processor in communication with the at least a sensor. The wearable medical device includes a memory communicatively connected to the processor. The memory contains instructions configured the processor to determine a submersion status based on the sensor data. The processor is configured to receive ambient temperature data from the sensor data. The processor is configured to calculate, based on the submersion status and the ambient temperature data, an adjusted delivery aspect of a medication for the user. The processor is configured to deliver the adjusted delivery aspect of a medication for the user.

In another aspect, a system for adjusting medication delivery through a wearable medical device is presented. The system includes a drug delivery device affixed to a user. The drug delivery device includes at least a sensor configured to generate sensor data. The drug delivery device includes a processing unit configured to determine a submersion status based on the sensor data. The processing unit is configured to receive ambient temperature data from the sensor data. The processor is configured calculate an adjusted delivery aspect of a medication for the user based on the submersion status and the ambient temperature data. The processor is configured to deliver the adjusted delivery of a medication to the user from the drug delivery device.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

FIG. 1 illustrates a block diagram of an exemplary embodiment of a wearable medical device;

FIG. 2A-B illustrates exemplary embodiments of a wearable medical device with temperature sensors;

FIG. 3 illustrates an exemplary embodiment of a wearable medical device with a microphone and inertial sensors;

FIG. 4 illustrates an exemplary embodiment of a wearable medical device adhered to a user;

FIG. 5 illustrates a block diagram of a wearable injection device;

FIG. 6 illustrates a flowchart of determining a probability of showering through an accelerometer;.

FIG. 7 illustrates a flowchart of determining a probability of showering through a microphone;

FIG. 8 illustrates a flowchart of determining a probability of showering through a motion signature library;

FIGS. 9A-B illustrate exemplary embodiments of a wearable medical device with a water detection system;

FIG. 10 illustrates a flowchart of determining a location assessment;

FIG. 11 illustrates a flowchart of generating weighted scores for a final prediction;

FIG. 12 illustrates a flowchart for determining blood glucose metrics;

FIG. 13 illustrates a flowchart for updating an insulin on board algorithm;

FIG. 14 illustrates a block diagram of a machine learning module.

### DETAILED DESCRIPTION

In the following description, for the purposes of explanation, numerous specific details are set forth in order to provide a thorough understanding of the present invention. It will be apparent, however, that the present invention may be practiced without these specific details. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims.

At a high level, aspects of the present disclosure relate to wearable medical devices. Aspects of the present disclosure may be used to accommodate for fluctuations in insulin levels of a user due to temperatures of bodies of water. Aspects of the present disclosure may be used to predict if a user is showering and learn user behaviors for future predictions.

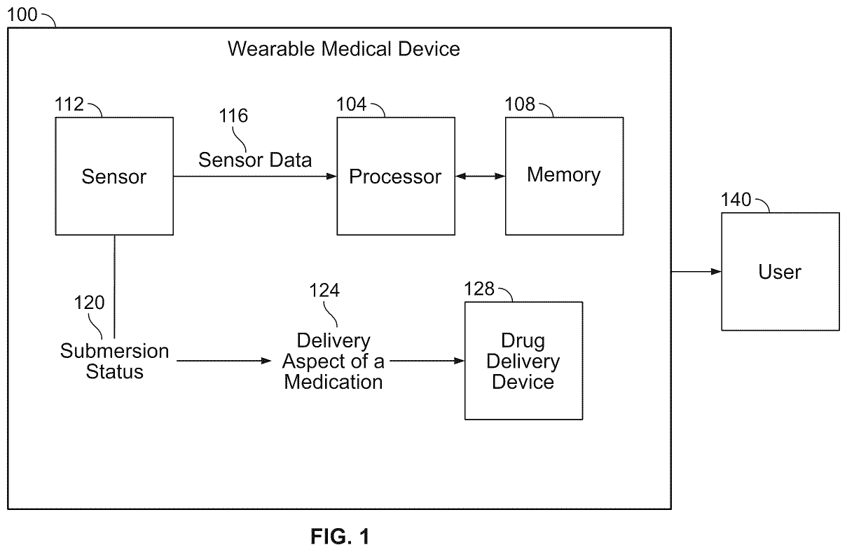

Referring now to FIG. 1, apparatus 100 for glycemic control is presented. Apparatus 100 may include processor 104 and/or memory 108. As used in this disclosure, “communicatively connected” means connected by way of a connection, an attachment, or a linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Still referring to FIG. 1, the processor 104 may include a computing device. The processor 104 may include any computing device as described in this disclosure, including without limitation a microcontroller, microprocessor, digital signal processor (DSP) and/or system on a chip (SoC) as described in this disclosure. The processor 104 may include, be included in, and/or communicate with a mobile device such as a mobile telephone or smartphone. The processor 104 may include a single computing device operating independently, or may include two or more computing device operating in concert, in parallel, sequentially or the like; two or more computing devices may be included together in a single computing device or in two or more computing devices. The processor 104 may interface or communicate with one or more additional devices as described below in further detail via a network interface device (not shown). Network interface device may be utilized for connecting the processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. The processor 104 may include but is not limited to, for example, a computing device or cluster of computing devices in a first location and a second computing device or cluster of computing devices in a second location. The processor 104 may include one or more computing devices dedicated to data storage, security, distribution of traffic for load balancing, and the like. The processor 104 may distribute one or more computing tasks as described below across a plurality of computing devices of computing device, which may operate in parallel, in series, redundantly, or in any other manner used for distribution of tasks or memory between computing devices. Apparatus 100 may be implemented using a “shared nothing” architecture in which data is cached at the worker, in an embodiment, this may enable scalability of the processor 104 and/or another computing device.

With continued reference to FIG. 1, the processor 104, and/or any other computing device as described throughout this disclosure, may be designed and/or configured to perform any method, method step, or sequence of method steps in any embodiment described in this disclosure, in any order and with any degree of repetition. For instance, the processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Apparatus 100 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Referring still to FIG. 1, the wearable medical device 100 may be configured to be affixed to the user 140. For instance, the wearable medical device 100 may include adhesive patches, clips, straps, locks, and/or other devices that may affix the wearable medical device 100 to the user 140. The wearable medical device 100 may include sensor. In some embodiments, the wearable medical device 100 may include a plurality of sensors 112. A “sensor” as used in this disclosure is a device capable of measuring a physical property and outputting data of the physical property. The sensor 112 may include, but is not limited to, thermometers, accelerometers, humidity sensors, gyroscopes, microphones, and/or other sensors. In some embodiments, a plurality of sensors 112 may be positioned on one or more sides of a housing of the wearable medical device 100. For instance, a plurality of sensors 112 may include a first sensor disposed on a left inner wall of a housing of the wearable medical device 100 and a second sensor disposed on a right inner wall of the housing of the wearable medical device 100. In some embodiments, a plurality of sensors 112 may include two or more sensors positioned in different locations of the wearable medical device 100, such as, but not limited to, a top portion, back portion, front portion, rear portion, bottom portion, side portion, and the like, of the wearable medical device 100. A location for each sensor of a plurality of sensors 112 may be selected based on a type of sensor of a plurality of sensors 112. As a non-limiting example, a microphone may be positioned on a top of an external surface of the wearable medical device 100 which may allow the microphone to pick up more soundwaves than if the microphone were positioned in a different location. A plurality of sensors 112 may, in some embodiments, be clustered together in a “sensor suite”. A sensor suite may include two or more sensors grouped together and/or in communication with one another. In embodiments, where a plurality of sensors 112 comprise a sensor suite, each sensor of the sensor suite may operate independently, in combination with one another, and the like. For instance, an accelerometer and a gyroscope of a plurality of sensors 112 may operate in combination to generate sensor data 116.

“Sensor data” as used in this disclosure is information generated from one or more sensors. The sensor data 116 may include, but is not limited to, temperatures, accelerations, velocities, humidity, sound waves, motions, and the like. The sensor data 116 may include ambient temperature data, such as, but not limited to, an immediate surrounding of wearable medical device 100. Ambient temperatures may include surrounding temperatures of wearable medical device 100 and/or user 140, such as within a radius of about up to 10 or more feet from the wearable medical device 100, without limitation. Ambient temperature data may include one or more values of temperature measured in, but not limited to, Fahrenheit (F), Celsius (C), Kelvin (K), and the like. In some embodiments, the sensor data 116 may include biological data. “Biological data” as used in this disclosure is information relating to a user's biology. Biological data may include, but is not limited to, heart rates, heart rhythms, blood pressure, blood glucose values, and the like. The sensor 112 may include one or more blood glucose monitors which may generate blood glucose values of a user. The sensor 112 may be in communication with the processor 104. The processor 104 may receive the sensor data 116 through a wired, wireless, and/or other connection from the plurality of sensor 112. In some embodiments, the processor 104 may store the sensor data 116 and/or other data in the memory 108. The processor 104 may be configured to communicate the sensor data 116 with an external computing device, such as, but not limited to, a server, desktop, laptop, smartphone, tablet, and the like. In some embodiments, the processor 104 may receive the sensor data 116 from an external computing device and/or other sensors that may be external to the wearable medical device 100. For instance and without limitation, the processor 104 may receive the sensor data 116 from a global positioning system (GPS) device, where the sensor data 116 may include locational data of the wearable medical device 100 and/or the user 140.

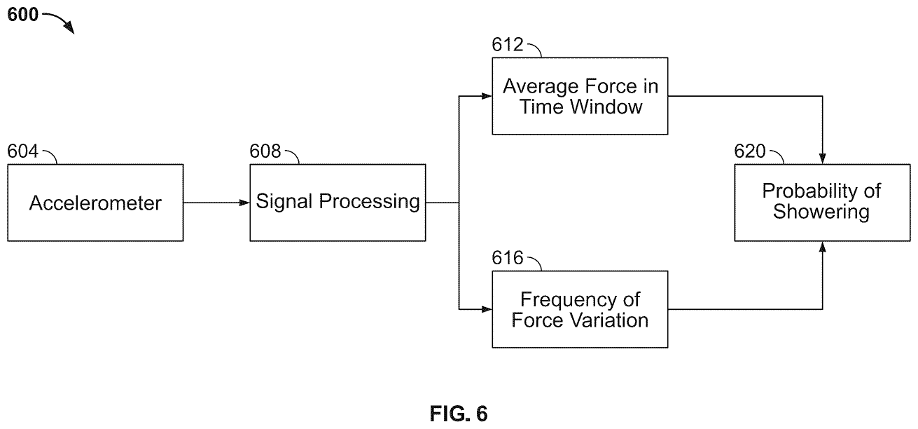

The processor 104 may be configured to generate submersion status 120 based on the sensor data 116. A “submersion status” as used in this disclosure is an indication of an amount of water in contact with an object and/or individual. For instance, the submersion status 120 may include one or more levels of submersion, such as, but not limited to, unsubmerged, humid, damp, wet, submerged, and the like. Each level of submersion may be determined by comparing the sensor data 116 to one or more submersion thresholds. A “submersion threshold” as used in this disclosure is a value or range of values that if met determines a submersion status. A submersion threshold may include one or more humidity values, such as, but not limited to, 40% humidity, 60% humidity, and the like. In some embodiments, the submersion threshold may include a combination of two or more sensor data types. As a non-limiting example, a submersion threshold may include a combination of a temperature value of 80 degrees Fahrenheit and a humidity value of 75%, at which the processor 104 may determine the submersion status 120 to be “wet”. Various sensor data types and corresponding submersion statuses may be described in further detail below with reference to FIGS. 6-9.

The processor 104, in some embodiments, may communicate the sensor data 116 to an external computing device and receive the submersion status 120 from the external computing device. In other embodiments, the processor 104 may calculate the submersion status 120 locally. In some embodiments, the processor 104 may adjust one or more operations of wearable medical device 100 based on submersion status 120. For instance, and without limitation, the processor 104 may determine and/or communicate errors and/or issues that may arise from wearable medical device 100 being in contact with water. The processor 104 may determine erroneous blood glucose readings, communications with other devices, and the like and flag and/or communicate any errors and/or issues with one or more computing devices. In some embodiments, the processor 104 may detect a presence of water through the submersion status 120 and may adjust processing power, antenna power of one or more antennas of wearable medical device 100, blood glucose measurement sensors of sensor 112, and the like.

The processor 104 may be configured to calculate delivery aspect of a medication 124. A “delivery aspect of a medication” as used in this disclosure refers to a quantity, timing, and/or duration of medication delivered through a drug delivery device. The delivery aspect of a medication 124 may include, without limitation, dosage amounts, dose timing, rate of dose changes, and the like. The delivery aspect of a medication 124 may include, but is not limited to, quantities, delivery time periods, medication types, and the like. In some embodiments, and without limitation, the delivery aspect of a medication 124 may include insulin delivery. The delivery aspect of a medication 124 may include parameters such as, but not limited to, total daily insulin (TDI) values, insulin-on board (IOB) values, insulin-to-carb ratios, correction factors, basal insulin rates, bolus dosing values, and the like. The processor 104 may calculate the delivery aspect of a medication 124 based on the submersion status 120 and/or the sensor data 116. For instance, the submersion status 120 may include a “humid” value and the sensor data 116 may include sound data generated from a microphone of the sensor 112 that may indicate the user 140 may be showering. The processor 104 may generate the delivery aspect of a medication 124 to accommodate a hot shower the user 140 may be standing in. As a non-limiting example, a user in a hot shower may experience an increase in a rate of insulin absorption, which may pose a risk for hypoglycemia. Continuing this example, the processor 104 may generate the delivery aspect of a medication 124 to include a reduced basal insulin dosage of about 20%. The processor 104 may use biological data in combination with other sensor data 116 to adjust the delivery aspect of a medication 124 in response to glycemic fluctuations of the user. A “glycemic fluctuation” as used in this disclosure is a change in parameters related to a blood glucose values of a user. Parameters related to a blood glucose value of a user may include, without limitation, average blood glucose values, HbA1c values, insulin-on board values, and the like. The processor 104 may determine the delivery aspect of a medication 124 and continually adjust the delivery aspect of a medication 124 based on one or more glycemic fluctuations of the user 140. For instance, continuing the showering example above, the sensor data 116 may show a glycemic fluctuation of the user 140 in the form of increased insulin on-board levels and may further reduce the basal insulin rate by about 10% for a total reduction of about 30%.

The processor 104 may, in some embodiments, compare ambient temperature data of the sensor data 112 with a thermal neutral threshold. A “thermal neutral threshold” as used in this disclosure is a value or range of values of temperatures that effect insulin absorption. For instance, a thermal neutral threshold may include a range of about, without limitation, 50 degrees F. to 80 degrees F., which may be a range of values that are deemed “safe” temperatures that may not severely effect insulin absorption. Temperatures below 50 degrees F. and/or higher than 80 degrees F. may effect insulin absorption in an individual. The processor 104 may compare ambient temperatures from the sensor data 112 to one or more values of a thermal neutral threshold. In some embodiments, the processor 104 may generate the adjusted delivery aspect of a medication 124 based on a comparison of one or more ambient temperatures to a thermal neutral threshold. The thermal neutral threshold values may be pre-programmed at processor 104. In some embodiments, the processor 104 may determine a thermal neutral threshold based on the sensor data 112, such as correlating ambient temperature data to biological data of the sensor data 112. The processor 104 may receive a thermal neutral threshold from one or more external computing devices, such as, but not limited to, smartphones, servers, desktops, laptops, and the like.

Still referring to FIG. 1, the wearable medical device 100 may include drug delivery device 128. A “drug delivery device” as used in this disclosure is a device capable of administering one or more chemicals to an individual. The drug delivery device 128 may include, without limitation, an insulin pump. The drug delivery device 128 may be positioned within a housing of the wearable medical device 100. In some embodiments, the drug delivery device 128 may be positioned on a bottom surface of the wearable medical device 100. The drug delivery device 128 may include an injection mechanism. An “injection mechanism” as used in this disclosure is an object or grouping of objects configured to administer medicine through a skin of an individual. For instance, an injection mechanism may include, without limitation, one or more needles, syringes, springs, cannulas, and/or other devices. An injection mechanism may be fluidically connected to a liquid reservoir. “Fluidically connected” as used in this disclosure refers to a form of communication between two objects through liquids. A fluidic communication may include, without limitation, one or more tubes, pipes, reservoirs, valves, and/or other devices. A “liquid reservoir” as used in this disclosure is a container of fluid. A liquid reservoir may be configured to contain one or more drugs, such as, but not limited to, insulin. A liquid reservoir may provide a fluid pathway for a medicine stored in the liquid reservoir to an injection mechanism, such as, but not limited to, one or more tubes. The drug delivery device 128 may be configured to administer the delivery aspect of a medication 124 through an injection mechanism to the user 140.

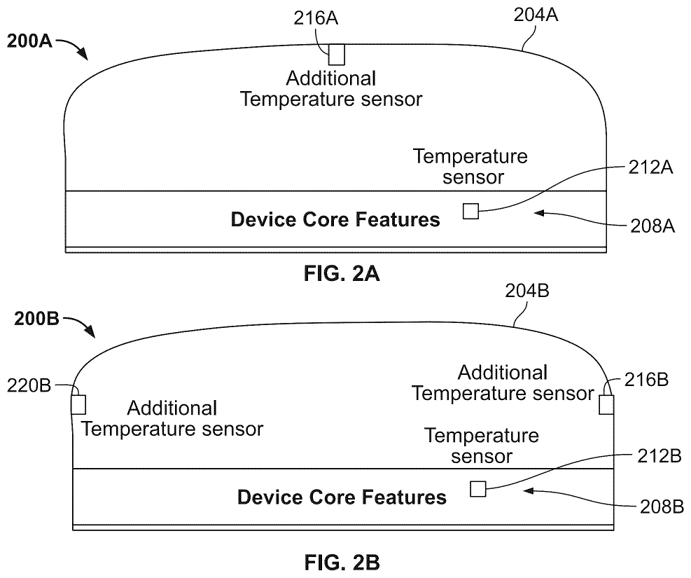

Referring now to FIG. 2A, an exemplary embodiment of a wearable medical device 200 with a temperature sensor is presented. The wearable medical device 200A may be the same as that of the wearable medical device 100 as described above with reference to FIG. 1. The wearable medical device 200A may include housing 204A. The housing 204A may be made of plastic and/or other materials. In some embodiments, the housing 204A may be curved, straight, and/or a combination thereof. The housing 204A may include a top curved portion connected to a bottom straight portion. The bottom straight portion of the housing 204A may include an affixing device, such as an adhesive patch, to affix to a user. The wearable medical device 200A may include device core features 208A. The device core features 208A. The device core features 208A may include one or more processors, injection mechanisms, liquid reservoirs, and the like. In some embodiments, the device core features 208A may include temperature sensor 212A. The temperature sensor 212A may include a thermometer or other temperature sensing device. In some embodiments, the wearable medical device 200A may include additional temperature sensor 216A. The additional temperature sensor 216A may be positioned at a top portion of the housing 204A. In some embodiments, the additional temperature sensor 216A may be positioned at a top middle portion of the housing 204A. The additional temperature sensor 216A may be in communication with a processor of the device core features 208A. The processor of the device core features 208A may utilize temperature readings from the additional temperature sensor 216A and/or the temperature sensor 212A. The processor of the device core features 208A may compare temperature readings from the additional temperature sensor 216A with temperature readings from the temperature sensor 212A to calculate a difference in temperature between a bottom portion of the housing 204 and a top portion of the housing 204A. The processor may determine a user's skin temperature, a processor's temperature, and the like through the temperature sensor 212A. The processor of the device core features may utilize the temperature readings of the additional temperature sensor 216A to determine and/or calculate an environmental temperature, such as an immediate surrounding of the wearable medical device 200A.

Referring now to FIG. 2B, an embodiment of the wearable medical device 200B with multiple additional temperature sensors is illustrated. The wearable medical device 200B may include housing 204B, the device core features 208B, and the temperature sensor 212B, as described above with reference to FIG. 2A. In some embodiments, the wearable medical device 200B may include two additional temperature sensors 216B and 220B. The additional temperature sensor 216B may be positioned at a side, such as a left side, of the housing 204B and the additional temperature sensor 220B may be placed opposite the additional temperature sensor 216B, such as at a right side of the housing 204B. The positioning of the additional temperature sensors 216B and 220B may allow for a more accurate temperature reading of an environment of the wearable medical device 200B. For instance, by having the additional temperatures sensors 216B and 220B on each side of the wearable medical device 200B, a processor of the device core features 208B may be able to determine an average environmental temperature across the wearable medical device 200B. The additional temperature sensors 216B and 220B may include thermometers and/or other temperature sensing devices. In some embodiments, each temperature sensor of the temperature sensor 212B, the additional temperature sensor 216B and the additional temperature sensor 220B may include a same sensor type. In other embodiments, each of the temperature sensors 212B, 216B, and 220B may each be different types of temperature sensors, such as, but not limited to, thermocouple, thermistor, infrared (IR), or other temperature sensor types. A processor of the device core features 208B may be configured to receive temperature readings from each of temperature sensor 212, additional temperature sensor 216B, and/or additional temperature sensor 220B. In some embodiments, a processor of the device core features 208 may determine environmental temperatures of the wearable medical device 200 through the additional temperature sensors 216B and 220B.

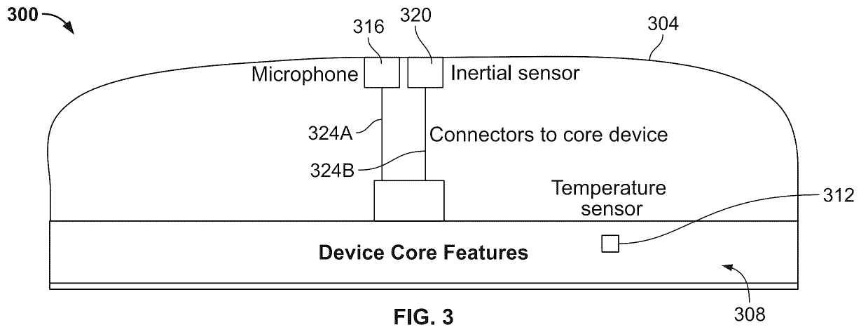

Referring now to FIG. 3, an exemplary embodiment of a wearable medical device 300 with a microphone is presented. The wearable medical device 300 may be the same as that of the wearable medical device 200A as described above with reference to FIG. 2A. The wearable medical device 300 may include housing 304, device core features 308, and/or temperature sensor 312, which may be the same as that of the housing 204A, device core features 208A, and temperature sensor 212A as described above with reference to FIG. 2A. The wearable medical device 300 may include microphone 316 and/or inertial sensor 320. The microphone 316 may include a diaphragm, coil, and/or permanent magnet. The microphone 316 may act as a transducer which may convert soundwaves into electrical energy. The microphone 316 may include, but is not limited to, dynamic microphones, condenser microphones, contact microphones, and the like. The microphone 316 may be configured to receive ambient and/or other soundwaves and generate sound data from the soundwaves. “Sound data” as used in this disclosure is information pertaining to vibrations in the air. Sound data may include, but is not limited to, loudness, frequencies, and the like. For instance, sound data may include data showing a soundwave at 70 dB. The inertial sensor 320 may be configured to transform mechanical energy, such as movements, into electrical signals. The inertial sensor 320 may be configured to detect movements of a user, movements of the wearable medical device 300, and the like. In some embodiments, the inertial sensor 320 may be configured to detect water drops that may come into contact with the wearable medical device 300. Each of the microphone 316 and/or the inertial sensor 320 may be connected to a processor of the device core features 308 through connectors 324A and/or 324B. The connectors 324A and 324B may be made of copper, aluminum, silver, and the like. The connectors 324A and 324B may include conductive wires having an insulating housing. In some embodiments, the connector 324A may connect the microphone 316 to a circuit and/or processor of the device core features 308 and the connector 324B may connect the inertial sensor 320 to the processor and/or circuit of the core device features 308. A processor of the device core features 308 may be configured to determine various water sources, such as, but not limited to, bathtubs, showers, swimming pools, rain, and the like.

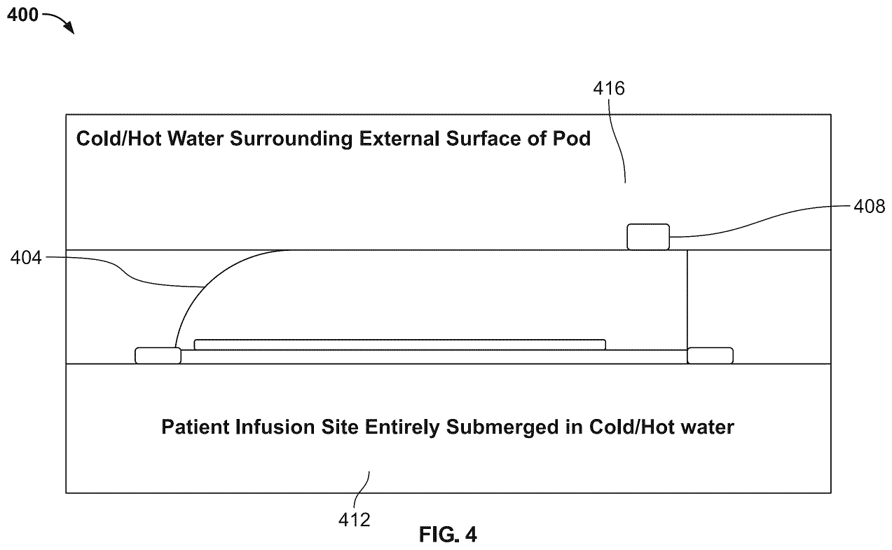

Referring now to FIG. 4, an exemplary embodiment of a submerged wearable medical device 400 is presented. The wearable medical device 400 may be the same as that of the wearable medical device 200A as described above with reference to FIG. 2A. The wearable medical device 400 may include housing 404 and temperature sensor 408, each of which may be the same as those described above with reference to FIG. 2A. In some embodiments, the temperature sensor 408 may be positioned on an exterior of house 404. For instance, and without limitation, the temperature sensor 408 may be positioned at a top right portion of the housing 404. In other embodiments, the temperature sensor 408 may be positioned at a central top portion of the housing 404. The wearable medical device 400 may be affixed to a user's skin 412. For instance, and without limitation, the wearable medical device 400 may be affixed to the user's skin 412 through an adhesive patch or other affixing mechanism. The temperature sensor 408 may be configured to measure temperature of water 416. The water 416 may be, without limitation, shower water, bath water, rainwater, swimming pool water, and the like. The temperature sensor 408 may determine a hot, cold, and/or other temperature of the water 416 and communicate any temperature readings to a processor of the wearable medical device 400. An algorithm of wearable medical device 400, such as an automated insulin delivery (AID) algorithm, may compare an expected blood glucose value to a change in temperature to accommodate fluctuations in blood glucose levels. For instance, an algorithm of wearable medical device 400 may flag a sudden change or sike in an upward/downward trend and may lower a micro bolus threshold and/or suspend delivery based on the flag.

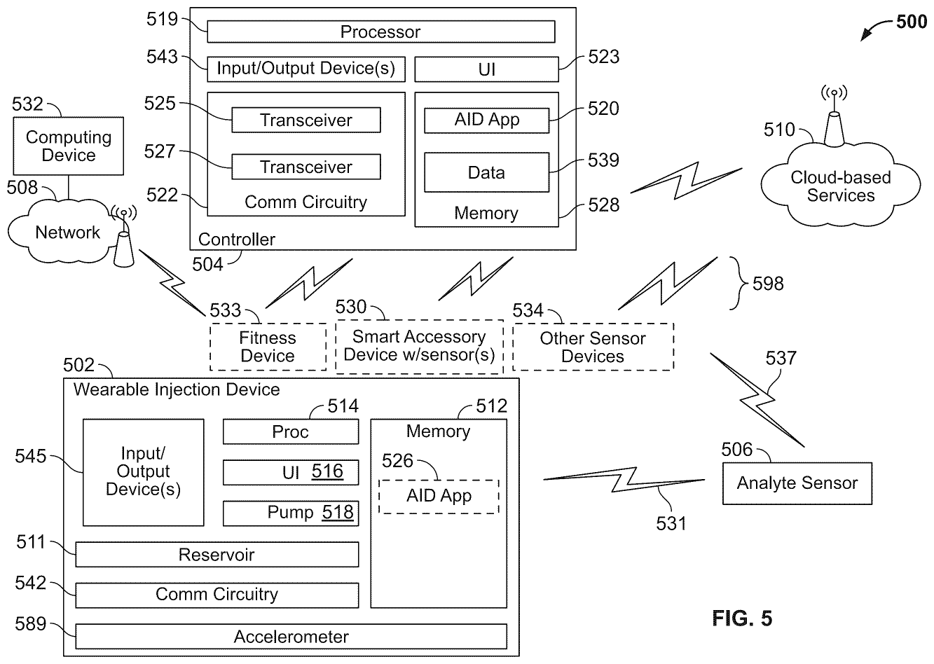

Referring now to FIG. 5, a block diagram of a drug delivery system 500 is illustrated. In some examples, the drug delivery system 500 is suitable for delivering insulin to a user in accordance with the disclosed embodiments. The drug delivery system 500 may include a wearable drug delivery device 502, a controller 504 and an analyte sensor 506. In addition, the drug delivery system may interact with a computing device 532 via a network 508 as well as obtain or contribute to cloud-based services 510.

Still referring to FIG. 5, the wearable drug delivery device 502 may be a wearable device that is worn on the body of the user. It may be similar to a wearable drug delivery and sensing system 100. The wearable drug delivery device 502 may be directly coupled to a user (e.g., directly attached to the skin of the user via an adhesive, or the like at various locations on the user's body, such as thigh, abdomen, or upper arm). In an example, a surface of the wearable drug delivery device 502 may include an adhesive to facilitate attachment to the skin of a user.

Still referring to FIG. 5, the wearable drug delivery device 502 may include a processor 514. The processor 514 may be implemented in hardware, software, or any combination thereof. The processor 514 may, for example, be a microprocessor, a logic circuit, a field programmable gate array (FPGA), an application specific integrated circuit (ASIC) or a microprocessor coupled to a memory. The processor 514 may maintain a date and time as well as be operable to perform other functions (e.g., calculations or the like). The processor 514 may be operable to execute an AID application 526 stored in the memory 512 that enables the processor 514 to direct operation of the wearable drug delivery device 502. The AID application 526 may control insulin delivery to the user per an AID algorithm. The memory 512 may store AID application settings for a user, such as specific factor settings, subjective insulin need parameter settings, and AID algorithm settings, such as maximum insulin delivery, insulin sensitivity settings, total daily insulin (TDI) settings and the like. The memory may also store data 529, such as drug delivery dosages, blood glucose measurement values, ketone measurement levels, and the like.

Still referring to FIG. 5, the analyte sensor 506 may be operable to collect physiological condition data, such as the blood glucose measurement values and a timestamp, ketone levels, heart rate, blood oxygen levels and the like that may be shared with the wearable drug delivery device 502, the controller 504 or both. For example, the communication circuitry 542 of the wearable drug delivery device 502 may be operable to communicate with the analyte sensor 506 and the controller 504 as well as the devices 530, 533 and 534. The communication circuitry 542 may be operable to communicate via Bluetooth®, Wi-Fi, a near-field communication standard, a cellular standard, or any other wireless protocol. In an example, the a wearable drug delivery and sensing system 100 may be a combination of the wearable drug delivery device 502 and the analyte sensor 506.

Still referring to FIG. 5, the input/output device(s) 545 may be one or more of a microphone, a speaker, a vibration device, a display, a push button, a touchscreen display, a tactile input surface, or the like. The input/output device(s) 545 may be coupled to the processor 514 and may include circuitry operable to generate signals based on received inputs and provide the generated signals to the processor 514. In addition, the input/output device(s) 545 may be operable to receive signals from the processor 514 and, based on the received signals, generate outputs via a respective output device.

Still referring to FIG. 5, the wearable drug delivery device 502 may include a reservoir 511. The reservoir 511 may be operable to store drugs, medications or therapeutic agents suitable for automated delivery, such as insulin, morphine, methadone, hormones, glucagon, glucagon-like peptide, blood pressure medicines, chemotherapy drugs, combinations of drugs, such as insulin and glucagon-like peptide, different peptides, or the like. A fluid path to the user may be provided via tubing and a needle/cannula (not shown). The fluid path may, for example, include tubing coupling the wearable drug delivery device 502 to the user (e.g., via tubing coupling a needle or cannula to the reservoir 511). The wearable drug delivery device 502 may be operable based on control signals from the processor 514 to expel the drugs, medications or therapeutic agents, such as insulin, from the reservoir 511 to deliver doses of the drugs, medications or therapeutic agents, such as the insulin, to the user via the fluid path. For example, the processor 514 by sending control signals to the pump 518 may be operable to cause insulin to be expelled from the reservoir 511.

Still referring to FIG. 5, there may be one or more communication links 598 with one or more devices physically separated from the wearable drug delivery device 502 including, for example, a controller 504 of the user and/or a caregiver of the user and/or a sensor 506. The analyte sensor 506 may communicate with the wearable drug delivery device 502 via a wireless communication link 531 and/or may communicate with the controller 504 via a wireless communication link 537. The communication links 531, 537, and 598 may include wired or wireless communication paths operating according to any known communications protocol or standard, such as Bluetooth, Wi-Fi, a near-field communication standard, a cellular standard, or any other wireless protocol.

Still referring to FIG. 5, the wearable drug delivery device 502 may also include a user interface (UI) 516, such as an integrated display device for displaying information to the user, and in some embodiments, receiving information from the user. For example, the user interface 516 may include a touchscreen and/or one or more input devices, such as buttons, knob or a keyboard that enable a user to provide an input.

Still referring to FIG. 5, in addition, the processor 514 may be operable to receive data or information from the analyte sensor 506 as well as other devices, such as smart accessory device 530, fitness device 533 or another wearable device 534 (e.g., a blood oxygen sensor or the like), that may be operable to communicate with the wearable drug delivery device 502. For example, fitness device 533 may include a heart rate sensor and be operable to provide heart rate information or the like.

Still referring to FIG. 5, the wearable drug delivery device 502 may interface with a network 508. The network 508 may include a local area network (LAN), a wide area network (WAN) or a combination therein and operable to be coupled wirelessly to the wearable drug delivery device 502, the controller, and devices 530, 533, and 534. A computing device 532 may be interfaced with the network 508, and the computing device may communicate with the insulin delivery device 502. The computing device 532 may be a healthcare provider device, a guardian's computing device, or the like through which a user's controller 504 may interact to obtain information, store settings, and the like. The AID application 520 may be operable to execute an AID algorithm and present a graphical user interface on the computing device 532 enabling the input and presentation of information related to the AID algorithm. The computing device 532 may be usable by a healthcare provider, a guardian of the user of the wearable drug delivery device 502, or another user.

Still referring to FIG. 5, the drug delivery system 500 may include an analyte sensor 506 for detecting the levels of one or more analytes of a user, such as blood glucose levels, ketone levels, other analytes relevant to a diabetic treatment program, or the like. The analyte level values detected may be used as physiological condition data and be sent to the controller 504 and/or the wearable drug delivery device 502. The sensor 506 may be coupled to the user by, for example, adhesive or the like and may provide information or data on one or more medical conditions and/or physical attributes of the user. The sensor 506 may be a continuous glucose monitor (CGM), ketone sensor, or another type of device or sensor that provides blood glucose measurements that is operable to provide blood glucose concentration measurements. The sensor 506 may be physically separate from the wearable drug delivery device 502 or may be an integrated component thereof. The analyte sensor 506 may provide the processor 514 and/or processor 519 with physiological condition data indicative of measured or detected blood glucose levels of the user. The information or data provided by the sensor 506 may be used to modify an insulin delivery schedule and thereby cause the adjustment of drug delivery operations of the wearable drug delivery device 502.

Still referring to FIG. 5, in the depicted example, the controller 504 may include a processor 519 and a memory 528. The controller 504 may be a special purpose device, such as a dedicated personal diabetes manager (PDM) device. The controller 504 may be a programmed general-purpose device that is a portable electronic device, such as any portable electronic device, smartphone, smartwatch, fitness device, tablet or the like including, for example, a dedicated processor, such as processor, a micro-processor or the like. The controller 504 may be used to program or adjust operation of the wearable drug delivery device 502 and/or the sensor 506. The processor 519 may execute processes to manage a user's blood glucose levels and that control the delivery of the drug or a therapeutic agent (e.g., a liquid drug or the like as mentioned above) to the user. The processor 519 may also be operable to execute programming code stored in the memory 528. For example, the memory 528 may be operable to store an AID application 520 for execution by the processor 519. The AID application 520 may be responsible for controlling the wearable drug delivery device 502, including the automatic delivery of insulin based on recommendations and instructions from the AID algorithm, such as those recommendations and instructions described herein.

Still referring to FIG. 5, the memory 528 may store one or more applications, such as an AID application 520, a voice control application 521, and data 539 which may be the same as, or substantially the same as those described above with reference to the insulin delivery device 502. In addition, the settings 521 may store information, such as drug delivery history, blood glucose measurement values over a period of time, total daily insulin values, and the like. The memory 528 may be further operable to store data and/or computer programs 539 and the like. In addition, the memory may store AID settings and parameters, insulin treatment program history (such as insulin delivery history, blood glucose measurement value history and the like. Other parameters such as insulin-on-board (IOB) and insulin-to-carbohydrate ratio (ICR) may be retrieved from prior settings and insulin history stored in memory. For example, the AID application 520 may be operable to store the AID algorithm settings, such as blood glucose target set points, insulin delivery constraints, basal delivery rate, insulin delivery history, bolus dosage history, wearable drug delivery device status, and the like. The memory 528 may also be operable to store data such as a food database for carbohydrate (or macronutrient) information of food components (e.g., grilled cheese sandwich, coffee, hamburger, brand name cereals, or the like). The memory 528 may be accessible to the AID application 520 and the voice control application 521.

Still referring to FIG. 5, the input/output device(s) 543 of the controller 504 may one or more of a microphone, a speaker, a vibration device, a display, a push button, a tactile input surface, touchscreen, or the like. The input/output device(s) 543 may be coupled to the processor 519 and may include circuitry operable to generate signals based on received inputs and provide the generated signals to the processor 519. In addition, the input/output device(s) 543 may be operable to receive signals from the processor 519 and, based on the received signals, generate outputs via one or more respective output devices, such as a speaker, a vibration device, or a display.

Still referring to FIG. 5, the controller 504 may include a user interface (UI) 523 for communicating visually with the user. The user interface 523 may include a display, such as a touchscreen, for displaying information provided by the AID application 520 or voice control application 521. The touchscreen may also be used to receive input when it is a touch screen. The user interface 523 may also include input elements, such as a keyboard, button, knob or the like. In an operational example, the user interface 523 may include a touchscreen display controllable by the processor 519 and be operable to present the graphical user interface, and in response to a received input (audio or tactile), the touchscreen display is operable present a graphical user interface related to the received input.

Still referring to FIG. 5, the controller 504 may interface via a wireless communication link of the wireless communication links 598 with a network, such as a LAN or WAN or combination of such networks that provides one or more servers or cloud-based services 510 via communication circuitry 522. The communication circuitry 522, which may include transceivers 527 and 525, may be coupled to the processor 519. The communication circuitry 522 may be operable to transmit communication signals (e.g., command and control signals) to and receive communication signals (e.g., via transceivers 527 or 525) from the wearable drug delivery device 502 and the analyte sensor 506. In an example, the communication circuitry 522 may include a first transceiver, such as 525, that may be a Bluetooth transceiver, which is operable to communicate with the communication circuitry 522 of the wearable drug delivery device 502, and a second transceiver, such as 527, that may be a cellular transceiver, a Bluetooth® transceiver, a near-field communication transceiver, or a Wi-Fi transceiver operable to communicate via the network 508 with computing device 532 or with cloud-based services 510. While two transceivers 525 and 527 are shown, it is envisioned that the controller 504 may be equipped with more or less transceivers, such as cellular transceiver, a Bluetooth transceiver, a near-field communication transceiver, or a Wi-Fi transceiver.

Still referring to FIG. 5, the cloud-based services 510 may be operable to store user history information, such as blood glucose measurement values over a set period of time (e.g., days, months, years), a drug delivery history that includes insulin delivery amounts (both basal and bolus dosages) and insulin delivery times, types of insulin delivered, indicated meal times, blood glucose measurement value trends or excursions or other user-related diabetes treatment information, specific factor settings including default settings, present settings and past settings, or the like.

Still referring to FIG. 5, other devices, like smart accessory device 530 (e.g., a smartwatch or the like), fitness device 533 and other wearable device 534 may be part of the drug delivery system 500. These devices may communicate with the wearable drug delivery device 502 to receive information and/or issue commands to the wearable drug delivery device 502. These devices 530, 533 and 534 may execute computer programming instructions to perform some of the control functions otherwise performed by processor 514 or processor 519. These devices 530, 533 and 534 may include user interfaces, such as touchscreen displays for displaying information such as current blood glucose level, insulin on board, insulin delivery history, or other parameters or treatment-related information and/or receiving inputs. The display may, for example, be operable to present a graphical user interface for providing input, such as request a change in basal insulin dosage or delivery of a bolus of insulin. Devices 530, 533 and 534 may also have wireless communication connections with the sensor 506 to directly receive blood glucose level data as well as other data, such as user history data maintained by the controller 504 and/or the wearable drug delivery device 502.

Still referring to FIG. 5, the user interface 523 may be a touchscreen display controlled by the processor 519, and the user interface 523 is operable to present a graphical user interface that offers an input of a subjective insulin need parameter usable by the AID application 520. The processor 519 may cause a graphical user interface to be presented on the user interface 523. Different examples of the graphical user interface may be shown with respect to other examples. The AID application 520 may generate instructions for the pump 518 to deliver basal insulin to the user or the like.

Still referring to FIG. 5, the processor 519 is also operable to collect physiological condition data related to the user from sensors, such as the analyte sensor 506 or heart rate data, for example, from the fitness device 533 or the smart accessory device 530. In an example, the processor 519 executing the AID algorithm may determine a dosage of insulin to be delivered based on the collected physiological condition of the user and a specific factor determined based on the subjective insulin need parameter. The processor 519 may output a control signal via one of the transceivers 525 or 527 to the wearable drug delivery device 502. The outputted signal may cause the processor 514 to deliver command signals to the pump 518 to deliver an amount of related to the determined dosage of insulin in the reservoir 511 to the user based on an output of the AID algorithm. The processor 519 may also be operable to perform calculations regarding settings of the AID algorithm as discussed as herein. Modifications to the AID algorithm settings provided via the voice control application 521, such as by the examples described herein, may be stored in the memory 528.

Referring now to FIG. 6, a flowchart of a process for determining a showering probability is presented. At step 604, data from an accelerometer is received. An accelerometer may be positioned in a wearable medical device, such as described above with reference to FIG. 1. In some embodiments, data from an accelerometer may include, but is not limited to, force, acceleration, and the like. The accelerometer may be configured to detect a movement of a wearable medical device and/or body part of a user. For instance, a wearable medical device including an accelerometer may be positioned on a user's arm. The data generated from the accelerometer may be indicative of a user's arm movements.

At step 608, the data from the accelerometer goes through signal processing. “Signal processing” as used in this disclosure is a technique used to convert data from one form to another form. Signal processing may include, but is not limited to, analog, continuous time, discrete time, digital, nonlinear, statistical, and/or other forms of signal processing. Accelerometer data may be filtered, such as between a frequency range of about 10 Hz or below, between about 10 Hz to about 20 Hz, and the like, without limitation. In some embodiments, a Fourier transform, fast Fourier transform, and/or other transform may be used. In some embodiments, data indicative of gravity may be subtracted from the accelerometer data by maintaining a running average. The accelerometer data may be processed to determine magnitudes of force in one or more frequency ranges. A wavelet transform may be used to process the accelerometer data. A wavelet transform may allow for determination of one or more magnitudes of one or more coefficients at different wavelet scales. The data from the accelerometer may be processed using any suitable signal processing technique, without limitation.

At step 612, the signal process outputs an average force in time window. An average force in time window may represent a mean force over a period of time that may be indicative of one or more actions. In some embodiments, an average force may include, without limitation, about 1 N to about 10 N. For instance, a mean force of 10 N over a span of 3 seconds may be indicative of turning a shower handle. In some embodiments, an average force may include about 0.25 N to about 2N over a span of 3 seconds, which may be indicative of water droplets from a shower head hitting a surface of a wearable medical device.

At step 616, the signal processing outputs a frequency of force variation. A frequency of force variation may include a rate of occurrence in a change of force measured. For instance, a higher frequency of force variation may be indicative of a user washing their head, using shampoo, and the like while a lower frequency of force variation may be indicative of a user resting.

At step 620, a probability of showering is determined based on the average force in time window and the frequency of force variation. A probability of showering may be calculated as a percentage, such as, but not limited to, 0% to 100%. In some embodiments, a probability of showering may be represented as a value from 0 to 1, such as, but not limited to 0.87. In some embodiments, a higher average force in time and a higher frequency of force variation may increase a probability of a user showering, while a lower average force in time and a lower frequency of force variation may decrease a probability of a user showering. In some embodiments, a lower average force in time and a higher frequency of force variation may increase a probability a user is showering, such as if a user is washing their hair. In some embodiments, a higher average force in time and a lower frequency of force variation may decrease a probability of a user showering.

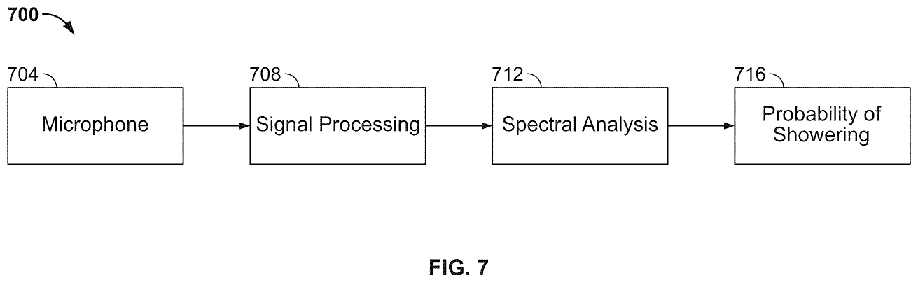

Referring now to FIG. 7, a flowchart of a process 700 for determining a probability of showering through sound data is presented. At step 704, a microphone generates sound data. The microphone may be part of or in communication with a wearable medical device, such as the wearable medical device as described above with reference to FIG. 1. In some embodiments, a plurality of microphones, such as a microphone array, may be used. Sound data generated from the microphone may include, without limitation, loudness, pitch/frequency, and/or other parameters of a soundwave.

At step 708, signal processing is performed on the sound data received from the microphone. Signal processing may include, but is not limited to, analog, continuous time, discrete time, digital, nonlinear, statistical, and/or other forms of signal processing. The data from the accelerometer may be processed using any suitable signal processing technique, without limitation.

At step 712, spectral analysis is performed on the processed signal. “Spectral analysis” as used in this disclosure is a technique used to calculate waves and/or oscillations in a set of data as a function of one or more independent variables. The spectral analysis may output a strength of components of a signal at different frequencies. The spectral analysis may be used to identify frequencies of a soundwave that may correlate to probabilities of showering.

At step 716, a probability of showering is calculated. The probability of showering may be calculated on a scale of 0 to 1, out of 100%, and the like. The probability of showering may be calculated based on the spectral analysis performed in step 712. For instance, certain sounds associated with showering, such as the sound of running water, may be identified through spectral analysis and compared to a threshold value, a threshold range, and the like.

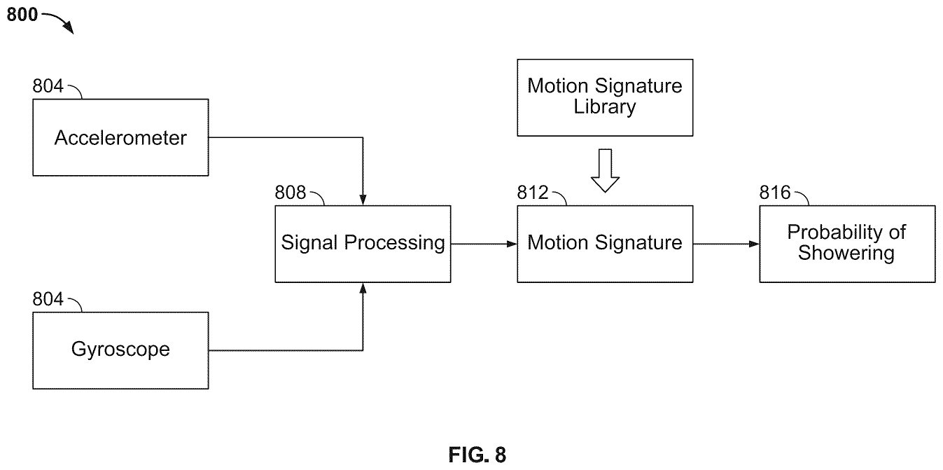

Referring now to FIG. 8, a flowchart for a process 800 of calculating a probability of showering through motion data is presented. At step 804, data is generated from an accelerometer and gyroscope. The accelerometer and gyroscope may be part of a wearable medical device, such as the wearable medical device as described above with reference to FIG. 1. The accelerometer may generate motion data such as, but not limited to, force, acceleration, and/or other data. The gyroscope may generate positioning data, such as, but not limited to, orientation, angular velocity, and the like.

At step 808, the motion data and positioning data generated from the accelerometer and the gyroscope goes through signal processing. Signal processing may include, but is not limited to, analog, continuous time, discrete time, digital, nonlinear, statistical, and/or other forms of signal processing. The data from the accelerometer may be processed using any suitable signal processing technique, without limitation.

At step 812, the motion signature is generated from the processed signal. The motion signature may include motion data and/or positioning data associated with an action. For instance, a motion signature may include a shampooing motion, hair washing motion, and the like. The motion signature generated from the processed signal may be compared to one or more motion signatures from a motion signature library. A motion signature library may be a database that stores a plurality of motion data and positioning data associated with a plurality of actions. For instance, the motion signature library may include motion data and/or positioning data associated with washing hair, scrubbing with a luffa, shaving legs, and the like.

At step 816, a probability of showering is calculated. The probability of showering may include a value out of 100, a percentage, a value between 0 and 1, and the like. The probability of showering may be calculated based on a comparison of the motion signature to one or more motion signatures of the motion signature library. In some embodiments, a threshold value may be used to indicate a probability of a user showering. For instance, the motion signature may align with a motion signature of shampooing of a motion signature library by about 70%, which may increase the probability the user is showering.

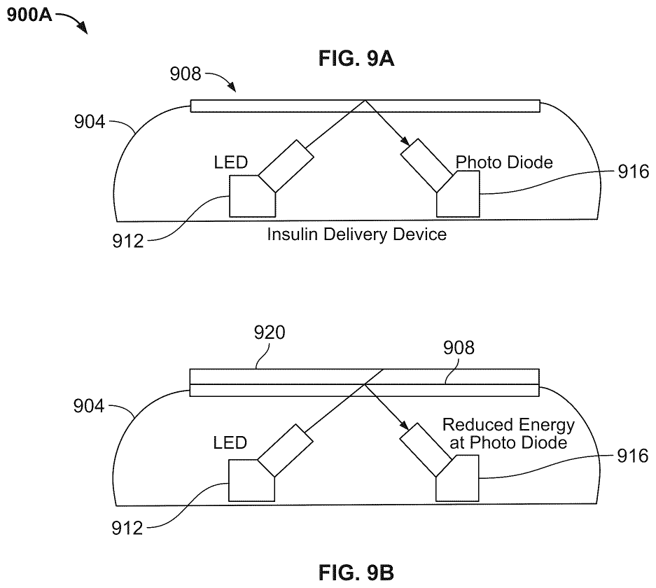

Referring now to FIG. 9A, a wearable medical device 900 with water detection capabilities is presented. The wearable medical device 900 may be the same as that of the wearable medical device 100 as described above with reference to FIG. 1. The wearable medical device 900 may include hosing 904. The housing 904 may include a top curved portion and a bottom straight portion. In some embodiments, the housing 904 may include reflective surface 908. The reflective surface 908 may be made of glass, plastic, and the like. In some embodiments, the reflective surface 908 may be configured to redirect photons. In some embodiments, the wearable medical device 900 may include LED 912. The LED 912 may be configured to transmit a laser towards the reflective surface 908. The LED 912 may be angled to accommodate a refractive index of the reflective surface 908. The reflective surface 908 may redirect the laser towards the photo diode 916. In cases where the wearable medical device 900 is dry, most or all of the laser may be redirected to the photo diode 916. A processor of the wearable medical device 900 may determine the wearable medical device 900 is dry based on the power of the laser received at the photo diode 916.

Referring now to FIG. 9B, a wearable medical device 900 in contact with a water surface is presented. The wearable medical device 900 may be the same as that described above with reference to FIG. 9A. The reflective surface 908 and/or the housing 904 may be in contact with the water surface 920. The water surface 920 may include a layer of water that may be in contact with the housing 904 and/or the reflective surface 908. In some embodiments, the LED 912 may transmit a laser to the reflective surface 908. The laser may hit the water surface 920 and redirect some of its energy away from the photo diode 916, changing the refractive index of the reflective surface 908. The remaining laser that hits the photo diode 916 may have a reduced power and/or energy. A processor of the wearable medical device 900 may determine, based on the reduced energy received at the photo diode 916, that the wearable medical device 900 is in contact with water 920. Various energy differences may be used to calculate degrees of water contact, such as, but not limited to, humid, damp, wet, and/or submerged, without limitation.

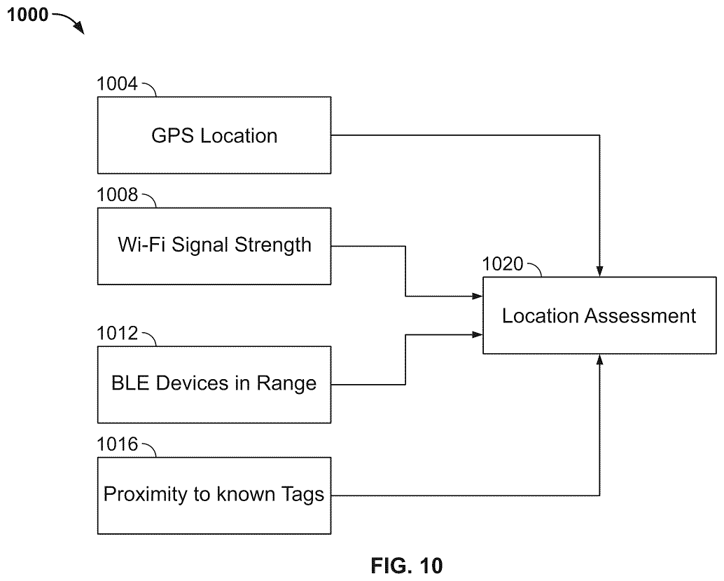

Referring now to FIG. 10, a flowchart for a process of determining a location assessment is provided. At step 1004, a GPS location is obtained. A GPS location may include a location of a user and/or wearable medical device of a user. The wearable medical device may include a GPS component that may interact with one or more other GPS components. In some embodiments, the GPS location may be determined in a latitude and/or longitude.

At step 1008, a Wi-Fi signal strength is obtained. A Wi-Fi signal strength may be obtained through one or more Wi-Fi components of a wearable medical device. The Wi-Fi signal strength may be relative to one or more Wi-Fi spots, such as routers, beacons, and the like. In some embodiments, the Wi-Fi signal strength may be measured in terms of power, such as milliwatts (mW), decibels, and the like. As a non-limiting example, −40 dBm may be equal to 0.0001 mW. In some embodiments, the Wi-Fi signal strength may be measured in a received signal strength indicator (RSSI) value. An RSSI value may include a value on a scale of 0-60, 0-255, and the like.

At step 10012, a Bluetooth signal is obtained. In some embodiments, a plurality of Bluetooth signals may be obtained. The Bluetooth signals may be transmitted between a Bluetooth component of a wearable medical device and one or more other Bluetooth devices, such as, but not limited to, smartphones, tablets, beacons, and the like. Bluetooth signal strength may be measured in dBm similarly to that of the Wi-Fi signal as described above with reference to step 1008.

At step 10014, a proximity to one or more tags is obtained. A tag may include, but is not limited to, a near field communication (NFC), radio frequency identification (RFID), Bluetooth tags, Wi-Fi tags, and the like. Tags may be positioned around areas of interest. For instance, in the case of determining if a user is showering, tags may be placed around a bathroom, shower head, shower curtain rod, and the like, without limitation.

At step 10018, a location assessment is generated based on the GPS location, Wi-Fi signal strength, Bluetooth signal, and/or proximity to one or more tags obtained through the previous steps at 1004, 1008, 10012, and 10014. The location assessment may include a position of a user and/or a wearable medical device. The location assessment may be relative to one or more locations. For instance, the location assessment may include a location of “bathroom”, “pool”, “hot tub”, and the like, without limitation. The location assessment may include a distance to one or more points of interest. As a non-limiting example, the location assessment may include a distance of 3 feet from a user and/or wearable medical device to a shower. The location assessment may be calculated locally with respect to a wearable medical device. In other embodiments, each component of the location assessment may be communicated to an external computing device, to which the external computing device may calculate the location assessment and communicate the location assessment to the wearable medical device and/or other computing device. In some embodiments, the location assessment may include a confidence score. A confidence score may include a value out of a range of 0 to 1, a percentage, and the like, which may be indicative of an accuracy of the location assessment. For instance, and without limitation, the location assessment may include a label of “swimming pool” with a relative distance to a swimming pool of about 5 feet. A confidence score of the location assessment above may include a value of about 0.87, or 87%.

Each of the GPS location, Wi-Fi signal strength, Bluetooth signal, and/or proximity to tags may be weighted in determining a final location assessment. For instance, the GPS location may have a weight of about 0.5, the Wi-Fi signal strength may have a weight of about 0.2, the Bluetooth signal strength may have a weight of about 0.15, and the proximity to tags may have a weight of about 0.15, which may total 1, without limitation. Each weight of each component of the location assessment may be updated and/or adjusted based on various factors, such as a quantity of tags, a latency of a Wi-Fi signal, an accuracy of a GPS location, and the like.

In some embodiments, a machine learning model, such as a supervised, unsupervised, or other machine learning model may be used to determine a final location assessment. A machine learning model may be trained with training data correlating GPS locations, Wi-Fi signal strengths, and Bluetooth signal strengths to final location assessments. Training data may be received through user input, external computing devices, and/or previous iterations of processing. A machine learning model may be configured to input a GPS location, Wi-Fi signal strength, and/or Bluetooth signal and output a final location assessment. In some embodiments, a machine learning model may be trained with training data correlating one or more priors of signal strength of GPS, Wi-Fi, Bluetooth, or other signals to a final location assessment. A machine learning model may be configured to match a signal strength of a GPS, Wi-Fi, Bluetooth, or other signal with one or more signal priors. In some embodiments, a machine learning model may be configured to determine a final location assessment based on a detection of a shower. For instance, a machine learning model may be trained with training data correlating GPS, Wi-Fi, and/or Bluetooth signals to one or more shower locations. A machine learning model may use a shower location prediction to produce a final location assessment. For instance, and without limitation, a machine learning model may compare a GPS signal location to a proximity to a determine shower location and output a final location assessment of “bathroom” based on the proximity distance, such as 2 feet.

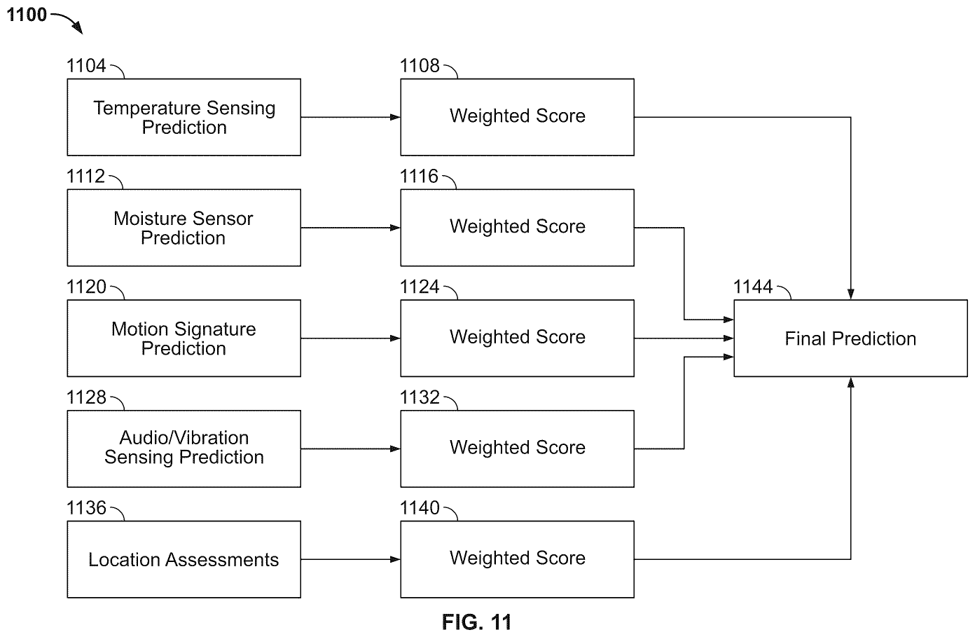

Referring now to FIG. 11, a flowchart for a process of sensor fusion to calculate a probability of showering is presented. At step 1104, a temperature sensing prediction is obtained. The temperature sensing prediction may include a reading from one or more temperature sensors of a wearable medical device. In some embodiments, a temperature sensor may be external to a wearable medical device and may communicate temperature data to the wearable medical device wirelessly. For instance, and without limitation, a temperature sensor with Bluetooth capabilities may be placed inside a bathroom near a shower. The temperature sensor in the above instance may communicate changes in temperature with a wearable medical device and/or other computing device. The temperature data of the temperature sensor external to the wearable medical device may be used in combination with one or more other temperature readings of one or more temperature sensors of the wearable medical device to predict an environmental temperature. An environmental temperature sensor/tag may include an average heat energy surrounding a wearable medical device and/or a user. For instance, an environmental temperature may include, without limitation, 60 degrees Fahrenheit (F), 70 degrees F., 80 degrees F., 90 degrees F., and the like.

At step 1108, a weighted score is assigned to the temperature sensing prediction. The weighted score may be a value between 0 to 1, a ratio, percentage, and/or other value. For instance, the weighted score of the temperature sensing prediction may be 0.2, without limitation. The weighted score of the temperature sensing prediction may be updated and/or adjusted based on a plurality of factors, such as, but not limited to, quantity of temperature sensors, location of temperature sensors, accuracy of temperature sensors, and the like. As a non-limiting example, a wearable medical device may include 5 highly sensitive temperature sensors. In this non-limiting example, due to the quantity of the 5 highly sensitive temperature sensors, a weighted score of the temperature sensing prediction may be a value of about 0.3, which may be higher than that of a wearable medical device having only 2 or less temperature sensors.

At step 1112, a moisture sensor prediction is obtained. The motion sensor prediction may be obtained through one or more moisture sensors of a wearable medical device and/or one or more moisture sensors that may be external to the wearable medical device. The moisture sensor prediction may include a value of about, but not limited to, a value out of 10, 100, and the like. For instance, the moisture sensor prediction may include a value of about 0.6, which may indicate a relative wetness of about 60% with 100% being completely wet and 0% being completely dry.

At step 1116, a weighted score is applied to the moisture sensor prediction. The weighted score may include a value between 0 to 1, a value out of 100, a percentage, and the like. The weighted score may be determined through a plurality of factors, such as, without limitation, quantity of moisture sensors, moisture sensor prediction values, and the like. For instance, a moisture sensor prediction value of about 0.8 may be given a weighted score of about 0.35 due to the higher moisture sensor prediction value.

At step 1120, a motion signature prediction is obtained. The motion signature prediction may be obtained through one or more accelerometers, gyroscopes, inertial measurement units, and/or other sensor types of a wearable medical device and/or other device. The motion signature prediction may be obtained as discussed above with reference to FIG. 8, without limitation. The motion signature prediction may include one or more motion signatures, which may include, without limitation, “shampooing”, “shaving”, “hair washing”, “body scrubbing”, and the like.

At step 1124, a weighted score is applied to the motion signature prediction. The weighted score may include a value from 0 to 1, out of 100, a percentage, and the like, without limitation. In some embodiments, the weighted score may be determined based on the type of motion signature prediction obtained. For instance, a motion signature of “hair washing” may have a higher weighted score than a motion signature of “shower knob turning”.

At step 1128, an audio and/or vibration sensing prediction is obtained. The audio and/or vibration sensing prediction may be obtained through one or more microphones, accelerometers, and the like, which may be part of a wearable medical device and/or external to the wearable medical device. For instance, a wearable medical device may include a microphone and accelerometer. In other embodiments, one or more microphones may be placed around positions of interest and may convey or otherwise communicate audio/sound data to a wearable medical device and/or computing device. In some embodiments, a wearable medical device may receive vibration sensing from an accelerometer internal to the wearable medical device. In other embodiments, a wearable medical device and/or other computing device may receive data from an accelerometer of a smartwatch or other accessory. The audio prediction may be obtained as described above with reference to FIG. 7, without limitation. The vibration prediction may be obtained as described above with reference to FIG. 6, without limitation.

At step 1132, a weighted score is applied to the audio and/or vibration sensing prediction. The weighted score may include a value out of 100, from 0 to 1, a percentage, and the like. In some embodiments, the weighted score may be adjusted and/or updated based on the type of audio and/or vibration sensing prediction obtained. For instance, an audio sensing prediction of “shower water running” may have a higher weighted value than that of an audio sensing prediction of “ambient noise”. Likewise, in some embodiments, a vibration sensing prediction of “washing hair” may have a higher weighted value than that of a vibration sensing prediction of “arms resting”.

At step 1136, a location assessment is obtained. The location assessment may be obtained as described above with reference to FIG. 10, without limitation. The location assessment may include a label, such as, but not limited to, “bathroom”, “hot tub”, “shower”, and the like. The location assessment may include distances relative to one or more objects/points of interest. For instance, the location assessment may include a distance of about 3 ft from a user to a shower.

At step 1140, a weighted score is applied to the location assessment. The weight score may include a value out of 100, a value between 0 to 1, a percentage, and the like. The weighted score may be updated and/or adjusted based on a label of the location assessment, a confidence score of the location assessment, and the like. For instance, a label of the location assessment of “shower” with a confidence score of about 0.94 may have a higher weighted score than a label of “kitchen” with a confidence score of about 0.57.

At step 1144, a final prediction is calculated. The final prediction may be calculated based on the temperature sensing prediction, moisture sensor prediction, motion signature prediction, audio and/or vibration sensing predictions, location assessments, and/or other inputs. The final prediction may include a label, such as, without limitation, “near a shower”, “showering”, “leaving a shower”, and the like. The final prediction may be communicated to a wearable medical device and/or other computing device. In some embodiments, the final prediction may include a confidence score. For instance, a confidence score of the final prediction may include a value out of 100, between 0 to 1, and the like. The confidence score may include, for example, a value of about 0.89.

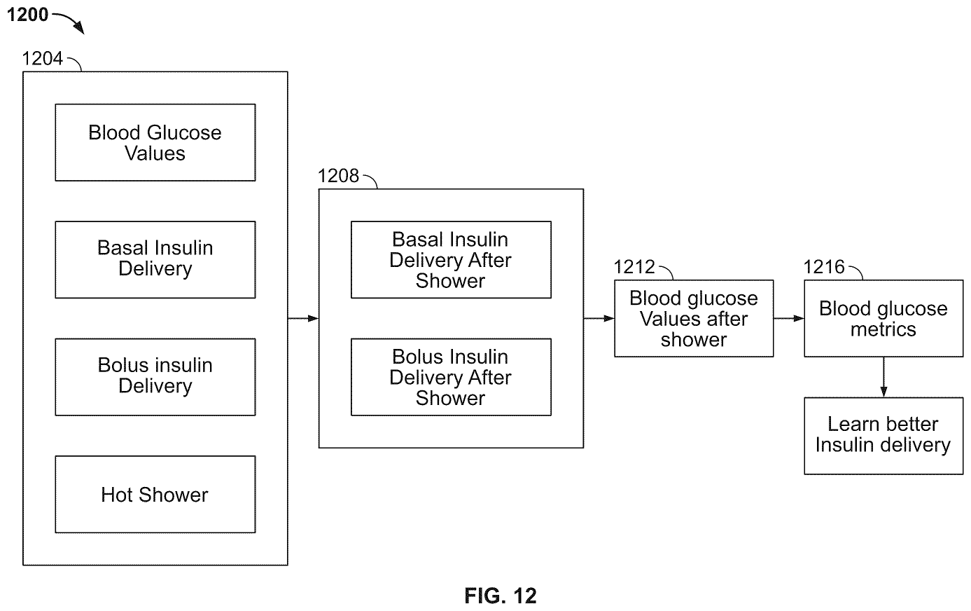

Referring now to FIG. 12, a flowchart for a process of adjusting a delivery aspect of a medication is presented. At step 1204, blood glucose values, basal insulin delivery values, bolus insulin delivery values, and/or other values may be obtained during a user taking a hot shower. Blood glucose values may include values from about 80 mg/dL to about 140 mg/dL. In some embodiments, the blood glucose values may be less than 80 mg/dL or greater than 140 mg/dL. The blood glucose values may be obtained through a blood glucose sensor, such as a blood glucose sensor of a wearable medical device. The basal insulin delivery values may include ranges of about, but not limited to, 2 units per hour to 10 units per hour. In some embodiments, the basal insulin delivery values may be less than 2 units per hour or more than 10 units per hour, without limitation. The bolus insulin delivery values may include a range of about 1 unit to 25 units. In some embodiments, the bolus insulin delivery values may be less than 1 unit or greater than 25 units. Each of the blood glucose values, basal insulin delivery values, and/or bolus insulin delivery values may be obtained through a wearable medical device, such as the wearable medical device described above in FIG. 1.

At step 1208, basal insulin delivery after a shower is obtained. The basal insulin delivery after a shower may include a value between about 2 units per hour to about 10 units per hour. In some embodiments, the basal insulin delivery after a shower may include a value below 2 units per hour or above 10 units per hour. The basal insulin delivery values after a user takes a hot shower may be received from a wearable medical device, such as the wearable medical device described above with reference to FIG. 1.

At step 1212, blood glucose values after a shower are obtained. The blood glucose values may be obtained through a blood glucose sensor, continuous blood glucose monitor (CGM), and the like. Blood glucose values of a user may fluctuate due to hot temperatures of a shower and/or the basal/bolus insulin deliveries. For instance the blood glucose values may deplete during/after a hot shower due to a higher absorption rate of insulin.

At step 1214, blood glucose metrics are obtained. The blood glucose metrics may be obtained through one or more sensors of a wearable medical device. In some embodiments, the blood glucose metrics may be obtained from a CGM or other device that may be external to a wearable medical device. The blood glucose metrics may include, without limitation, average change in blood glucose, changes in blood glucose over time, and the like.

At step 1216, an algorithm, such as an Automated Insulin Delivery (AID), algorithm, may learn from the blood glucose metrics obtained at step 1214 and/or from other data obtained throughout process 1200. The algorithm may learn to provide better deliveries of medication, such as through timing of bolus insulin deliveries, dosages and/or rates of basal insulin deliveries, and the like. In some embodiments, a machine learning model may be used. A machine learning model may be trained with training data correlating blood glucose values to blood glucose metrics. Training data may be received through user input, external computing devices, and/or previous iterations of processing. A machine learning model may be configured to input blood glucose values, basal insulin delivery values, bolus insulin delivery values, and the like, and output one or more blood glucose metrics. A machine learning model may use one or more blood glucose metrics as an error metric and may learn to minimize the error metric, which may allow for better insulin delivery.

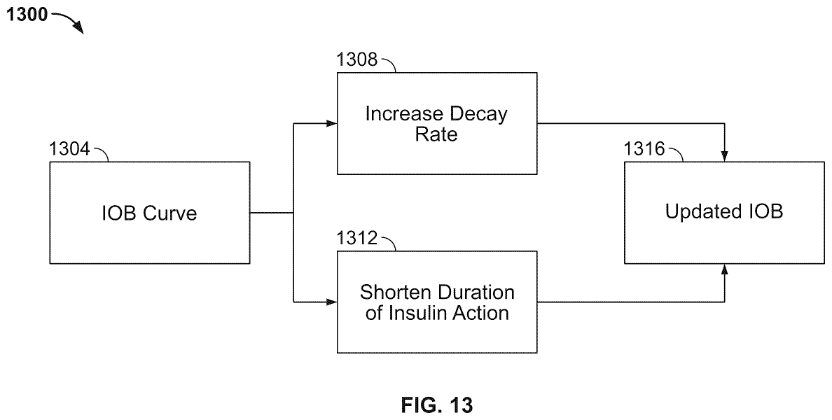

Referring now to FIG. 13, a flowchart for a process of adjusting a delivery of medication is presented. At step 1304, an insulin on board (IOB) curve is generated. The IOB curve may be representative of levels of insulin in a user's body before, during, and after coming into contact with water, such as a hot shower, swimming pool, and the like. In some embodiments, the IOB curve may show a faster absorption of insulin, where a remaining fraction of insulin may be less than a typical baseline of insulin.

At step 1308, process 1300 generates an increase in a decay rate of insulin decay rate. An increase in a decay rate of insulin may be generated in response to an increased absorption rate of insulin of a user while the user is in contact with water, such as a hot shower. For instance, a decay rate of insulin may increase from about 10% reduction in IOB levels over an hour to about 15% reduction in IOB levels over an hour, without limitation.

At step 1312, process 1300 generates a shortened duration of insulin action. A shorten duration of insulin action (DIA) may be generated in response to an increase in absorption rates of insulin of a user, such as when a user is taking a hot shower, without limitation. In some embodiments, the DIA may be shortened by, but not limited to, 1 hour, 2 hours, 3 hours, and the like. A shortening of the DIA may allow for more insulin to be delivered after a hot shower, which may account for the increased rate of absorption of insulin levels of a user.

At step 1316, process 1300 updates the IOB curve. The IOB curve may be updated based on the increased decay rate and/or the shortened DIA calculated above at steps 1312 and 1308. In some embodiments, the updated IOB may be generated to account for increased absorption rates of insulin of a user taking a hot shower.

While the examples are described with primarily with reference to insulin for ease of discussion, the disclosed systems, devices and techniques are more broadly applicable to a medication of which there are a variety. Examples of medications may include any drug in liquid form capable of being administered by a drug delivery device via a subcutaneous cannula, including, for example, insulin, glucagon-like peptide-1 (GLP-1), pramlintide, glucagon, co-formulations of two or more of GLP-1, and pramlintide; as well as pain relief drugs, such as opioids or narcotics (e.g., morphine, or the like), methadone, arthritis drugs, hormones, such as estrogen and testosterone, Alzheimer drugs, blood pressure medicines, chemotherapy drugs, fertility drugs, or the like.

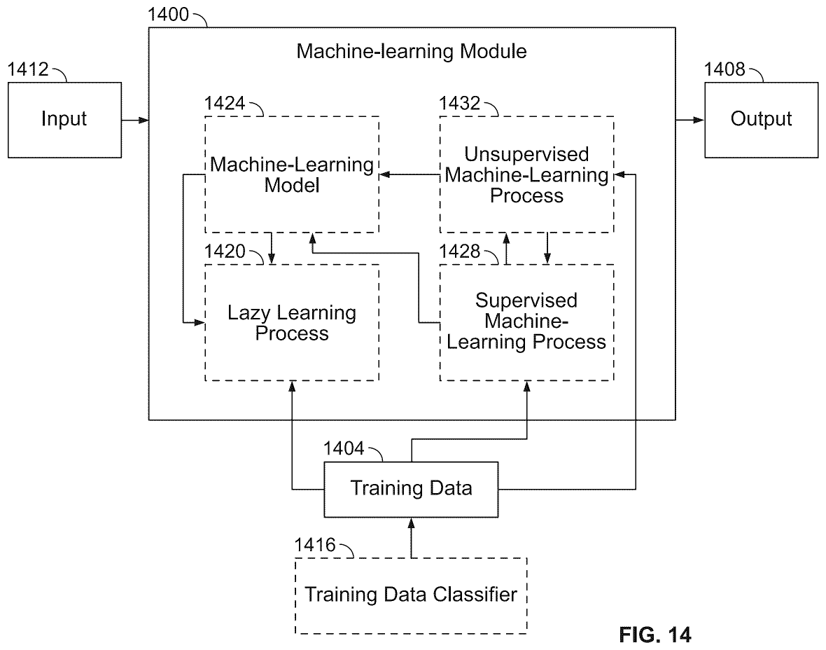

Referring to FIG. 14, an exemplary machine-learning module 1400 may perform machine-learning process(es) and may be configured to perform various determinations, calculations, processes and the like as described in this disclosure using a machine-learning process.

Still referring to FIG. 14, machine learning module 1400 may utilize training data 1404. For instance, and without limitation, training data 1404 may include a plurality of data entries, each entry representing a set of data elements that were recorded, received, and/or generated together. Training data 1404 may include data elements that may be correlated by shared existence in a given data entry, by proximity in a given data entry, or the like. Multiple data entries in training data 1404 may demonstrate one or more trends in correlations between categories of data elements. For instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 1404 according to various correlations. Correlations may indicate causative and/or predictive links between categories of data elements, which may be modeled as relationships such as mathematical relationships by machine-learning processes as described in further detail below. Training data 1404 may be formatted and/or organized by categories of data elements. Training data 1404 may, for instance, be organized by associating data elements with one or more descriptors corresponding to categories of data elements. As a nonlimiting example, training data 1404 may include data entered in standardized forms by one or more individuals, such that entry of a given data element in a given field in a form may be mapped to one or more descriptors of categories. Elements in training data 1404 may be linked to descriptors of categories by tags, tokens, or other data elements. Training data 1404 may be provided in fixed-length formats, formats linking positions of data to categories such as comma-separated value (CSV) formats and/or self-describing formats. Self-describing formats may include, without limitation, extensible markup language (XML), JavaScript Object Notation (JSON), or the like, which may enable processes or devices to detect categories of data.

With continued reference to refer to FIG. 14, training data 1404 may include one or more elements that are not categorized. Uncategorized data of training data 1404 may include data that may not be formatted or containing descriptors for some elements of data. In some embodiments, machine-learning algorithms and/or other processes may sort training data 1404 according to one or more categorizations. Machine-learning algorithms may sort training data 1404 using, for instance, natural language processing algorithms, tokenization, detection of correlated values in raw data and the like. In some embodiments, categories of training data 1404 may be generated using correlation and/or other processing algorithms. As a nonlimiting example, in a body of text, phrases making up a number “n” of compound words, such as nouns modified by other nouns, may be identified according to a statistically significant prevalence of n-grams containing such words in a particular order. For instance, an n-gram may be categorized as an element of language such as a “word” to be tracked similarly to single words, which may generate a new category as a result of statistical analysis. In a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 1404 to be made applicable for two or more distinct machine-learning algorithms as described in further detail below. Training data 1404 used by machine-learning module 1400 may correlate any input data as described in this disclosure to any output data as described in this disclosure, without limitation.

Further referring to FIG. 14, training data 1404 may be filtered, sorted, and/or selected using one or more supervised and/or unsupervised machine-learning processes and/or models as described in further detail below. In some embodiments, training data 1404 may be classified using training data classifier 1416. Training data classifier 1416 may include a classifier. A “classifier” as used in this disclosure is a machine learning model that sorts inputs into one or more categories. Training data classifier 1416 may utilize a mathematical model, neural net, or program generated by a machine learning algorithm. A machine learning algorithm of training data classifier 1416 may include a classification algorithm. A “classification algorithm” as used in this disclosure is one or more computer processes that generate a classifier from training data. A classification algorithm may sort inputs into categories and/or bins of data. A classification algorithm may output categories of data and/or labels associated with the data. A classifier may be configured to output a datum that labels or otherwise identifies a set of data that may be clustered together. Machine-learning module 1400 may generate a classifier, such as training data classifier 1416 using a classification algorithm. Classification may be performed using, without limitation, linear classifiers such as without limitation logistic regression and/or naive Bayes classifiers, nearest neighbor classifiers such ask-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 1416 may classify elements of training data to one or data categories, such as accelerometer data, GPS data, Bluetooth signal data, temperature data, and the like.

Still referring to FIG. 14, machine-learning module 1400 may be configured to perform a lazy-learning process 1420 which may include a “lazy loading” or “call-when-needed” process and/or protocol. A “lazy-learning process” may include a process in which machine learning is performed upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 1404. Heuristic may include selecting some number of highest-ranking associations and/or training data 1404 elements. Lazy learning may implement any suitable lazy learning algorithm, including without limitation a K-nearest neighbors algorithm, a lazy naive Bayes algorithm, or the like. Persons skilled in the art, upon reviewing the entirety of this disclosure, will be aware of various lazy-learning algorithms that may be applied to generate outputs as described in this disclosure, including without limitation lazy learning applications of machine learning algorithms as described in further detail below.

Still referring to FIG. 14, machine-learning processes as described in this disclosure may be used to generate machine-learning models 1424. A “machine-learning model” as used in this disclosure is a mathematical and/or algorithmic representation of a relationship between inputs and outputs, as generated using any machine-learning process including without limitation any process as described above, and stored in memory. For instance, an input may be sent to machine-learning model 1424, which once created, may generate an output as a function of a relationship that was derived. For instance, and without limitation, a linear regression model, generated using a linear regression algorithm, may compute a linear combination of input data using coefficients derived during machine-learning processes to calculate an output. As a further non-limiting example, machine-learning model 1424 may be generated by creating an artificial neural network, such as a convolutional neural network comprising an input layer of nodes, one or more intermediate layers, and an output layer of nodes. Connections between nodes may be created via the process of “training” the network, in which elements from a training data 1404 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 14, machine-learning algorithms may include supervised machine-learning process 1428. A “supervised machine learning process” as used in this disclosure is one or more algorithms that receive labelled input data and generate outputs according to the labelled input data. For instance, supervised machine learning process 1428 may include sensor data as described above as inputs, confidence maps as outputs, and a scoring function representing a desired form of relationship to be detected between inputs and outputs. A scoring function may maximize a probability that a given input and/or combination of elements inputs is associated with a given output to minimize a probability that a given input is not associated with a given output. A scoring function may be expressed as a risk function representing an “expected loss” of an algorithm relating inputs to outputs, where loss is computed as an error function representing a degree to which a prediction generated by the relation is incorrect when compared to a given input-output pair provided in training data 1404. Persons skilled in the art, upon reviewing the entirety of this disclosure, will be aware of various possible variations of at least a supervised machine-learning process 1428 that may be used to determine relation between inputs and outputs. Supervised machine-learning processes may include classification algorithms as defined above.

Further referring to FIG. 14, machine learning processes may include unsupervised machine-learning processes 1432. An “unsupervised machine-learning process” as used in this disclosure is a process that calculates relationships in one or more datasets without labelled training data. Unsupervised machine-learning process 1432 may be free to discover any structure, relationship, and/or correlation provided in training data 1404. Unsupervised machine-learning process 1432 may not require a response variable. Unsupervised machine-learning process 1432 may calculate patterns, inferences, correlations, and the like between two or more variables of training data 1404. In some embodiments, unsupervised machine-learning process 1432 may determine a degree of correlation between two or more elements of training data 1404.

Still referring to FIG. 14, machine-learning module 1400 may be designed and configured to create a machine-learning model 1424 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of I divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 14, machine-learning algorithms may include, without limitation, linear discriminant analysis. Machine-learning algorithm may include quadratic discriminate analysis. Machine-learning algorithms may include kernel ridge regression. Machine learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine learning algorithms may include ensemble methods such as bagging meta-estimator, forest of randomized tress, AdaBoost, gradient tree boosting, and/or voting classifier methods. Machine learning algorithms may include neural net algorithms, including convolutional neural net processes.

Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts requesting the input of information such as weight or age. Similarly, a dosage amount of insulin may be received by the AP application or algorithm from a user via a user interface. Other open-loop actions may also be implemented by adjusting user settings or the like in an AP application or algorithm.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A wearable medical device, comprising:
a drug delivery device delivering a delivery aspect of a medication to a user;
at least one sensor configured to generate sensor data;
a processor in communication with the at least one sensor; and
a memory communicatively connected to the processor, the memory containing instructions configuring the processor to:
determine a submersion status based on the sensor data;
receive ambient temperature data from the sensor data;
calculate, based on the submersion status and the temperature data, an adjusted delivery aspect of the medication; and
deliver the adjusted delivery aspect of medication to the user from the drug delivery device.

2. The wearable medical device of claim 1, wherein the processor is further configured to compare the ambient temperature data to a thermal neutral threshold and adjust the adjusted delivery aspect of a medication to the user based on the comparison.

3. The wearable medical device of claim 1, wherein the processor is further configured to calculate the adjusted delivery aspect of the medication based on biological data of the sensor data.

4. The wearable medical device of claim 1, wherein the processor is further configured to receive humidity data from the sensor data and calculate the adjusted delivery aspect of medication based on the humidity data.

5. The wearable medical device of claim 1, wherein the processor is further configured to determine a submersion status of the user based on the sensor data and calculate the adjusted delivery aspect of a medication based on the submersion status.

6. The wearable medical device of claim 1, wherein the processor is further configured to receive motion data from the sensor data and calculate the adjusted delivery aspect of a medication based on the motion data.

7. The wearable medical device of claim 1, wherein the processor is further configured to receive locational data from the sensor data and calculate the adjusted delivery aspect of a medication based on the locational data.

8. The wearable medical device of claim 1, wherein the processor is further configured to generate a confidence map based on data received from the plurality of sensors and calculate the adjusted delivery aspect of a medication for the user based on the confidence map.

9. The wearable medical device of claim 1, wherein the processor is further configured to calculate a probability of a user showering.

10. The wearable medical device of claim 1, wherein the medication includes insulin.

11. A system for adjusting medication delivery through a wearable medical device, comprising:
a drug delivery device affixed to a user, wherein the drug delivery device comprises:
at least one sensor configured to generate sensor data; and
a processing unit in communication with the at least one sensor, wherein the processing unit is configured to:
determine a submersion status based on the sensor data;
receive ambient temperature data from the sensor data;
calculate, based on the submersion status and the temperature data, an adjusted delivery aspect of a medication; and
deliver the adjusted delivery aspect of a medication to the user from the drug delivery device.

12. The system of claim 11, wherein the processing unit is further configured to compare the ambient temperature data to a thermal neutral threshold and calculate the delivery aspect of a medication to the user based on the comparison.

13. The system of claim 11, wherein the processing unit is further configured to calculate the adjusted delivery aspect of the medication based on biological data of the sensor data.

14. The system of claim 11, wherein the processing unit is further configured to receive humidity data from sensor data and calculate the adjusted delivery aspect of a medication to the user based on the humidity data.

15. The system of claim 11, wherein the processing unit is further configured to determine a submersion status of the user based on the humidity data and calculate the adjusted delivery aspect of a medication based on the submersion status.

16. The system of claim 11, wherein the processing unit is further configured to receive motion data from the sensor data and calculate the adjusted delivery aspect of a medication based on the motion data.

17. The system of claim 11, wherein the processing unit is further configured to receive locational data from the sensor data and calculate the adjusted delivery aspect of a medication based on the locational data.

18. The system of claim 11, wherein the processing unit is further configured to generate a confidence map based on data received from the sensor data and calculate the adjusted delivery of a medication for the user based on the confidence map.

19. The system of claim 11, wherein the processing unit is further configured to calculate a probability of a user showering.

20. The system of claim 11, wherein the medication includes insulin.
